(19) United States
(12) Patent Application Publication
(10) Pub. No.: US 2009/0107510 A1
(43) Pub. Date: Apr. 30, 2009

(54) TWO-LAYER ENDOTRACHEAL TUBE CUFF FOR PREVENTION OF PNEUMONIA

(75) Inventors: Katrina Cornish, Casa Grande, AZ (US); Jali Williams, Phoenix, AZ (US)

Correspondence Address: HOLME ROBERTS & OWEN LLP, 16427 N. SCOTTSDALE RD., SUITE 300, SCOTTSDALE, AZ, 85254, US

(73) Assignee: Yulex Corp.

(21) Appl. No.: 11/926,708

(22) Filed: Oct. 29, 2007

(51) Int. Cl. A61M 16/04

(52) U.S. Cl. 128/207.15

(57) ABSTRACT

A novel two-layer endotracheal tube (ETT) cuff for the prevention of pneumonia is disclosed. The disclosed two-layer ETT comprises a standard HVLP cuff covered with a second layer of elastomeric material with a sterile gel inserted between the layers. The two-layer cuff forms no folds when inflated in the trachea and prevents leakage, substantially reducing the risk for pneumonia attributable to standard ETT cuffs.

### FIELD OF THE INVENTION

This invention relates in general to the extraction of biopolymers from plant materials and more specifically to a fast and efficient system for expanded extraction of biopolymers from plant species containing biopolymers such as polyisoprene (rubber).

### BACKGROUND OF THE INVENTION

Ventilator-associated pneumonia (VAP) is a common complication during mechanical ventilation; aspiration of bacteria colonized secretions across the endotracheal tube cuff into the lower airways is a major risk factor for VAP. Such aspiration occurs along longitudinal folds formed when the high-volume low-pressure endotracheal tube cuff is inflated in the trachea.

Endotracheal tube (ETT) cuffs were initially made of thick (around 500 μm) Hevea latex rubber and required a high inflation pressure (200-400 cm H₂O) to form an adequate tracheal seal. The pressure transmitted to the tracheal wall was difficult to estimate from the intracuff pressure and consequently overinflation of the cuff was common. In some studies, tracheal wall pressures were as high as 200 cmH₂O, greatly above the tracheal mucosal capillary pressure about 30 cmH₂O. Widespread use of those low-volume high-pressure cuffs resulted in frequent pressure related tracheal injury.

In the early 1970s, disposable ETT cuffs made of polyvinylchloride (PVC), designated as high-volume low-pressure (HVLP) inflatable cuffs, were introduced to overcome this problem and remain the standard today. PVC cuffs are inelastic and 1.5-2 times larger than the internal diameter (ID) of the trachea. The HVLP cuff fills the trachea without being stretched, transmitting the intracuff pressure entirely to the tracheal wall. When inflated to 30 cmH₂O, the HVLP cuff permits mechanical ventilation and preserves tracheal capillary perfusion, but invariably forms multiple longitudinal folds. Bacteria colonized oropharyngeal secretions and gastric contents can leak along the folds into the lower airways and the lungs, a major risk factor for ventilator-associated pneumonia (VAP).

Guayule is a desert shrub native to the southwestern United States and northern Mexico and which produces polymeric isoprene essentially identical to that made by Hevea rubber trees (e.g., Hevea brasiliensis) in Southeast Asia. As recently as 1910 it was the source of half of the natural rubber used in the U.S. Since 1946, however, its use as a source of rubber has been all but abandoned in favor of cheaper Hevea rubber and synthetic rubbers. Still, demand for natural rubber is expected to produce shortages of that material in the future and rubber prices are expected to rise significantly. Natural rubber having lower heat hysteresis is required for many kinds of tires and amounts to about 35% of U.S. rubber use.

As an alternative to synthetic rubber sources, attention is being directed to the production of hydrocarbons in plants such as guayule (Parthenium argentatum). Guayule normally yields one half ton to one ton of rubber per acre in cultivation when, after two years, the entire plant is harvested and processed. Guayule plants store latex in tiny inclusions in the bark, making harvest of the outer fibrous layers, or bagasse, of the plant, desirable.

### BRIEF DESCRIPTION OF THE FIGURES

FIG. 1A shows the tracheal tube cuff inflated at 20 cmH₂O of pressure in a cylindrical glass tube (ID 20 mm) with no fold and no dye leaking.

FIG. 1B illustrates the tracheal tube cuff according to the present disclosure with a high-volume low-pressure cuff, draped by a very thin, highly compliant guayule latex cuff.

FIGS. 1C and 1D illustrate a cross section of the tracheal tube cuff according to the present disclosure.

FIG. 2 graphically illustrates average leakage flow (ml/min) across the five endotracheal tube cuffs at cuff pressures of 20, 25, 30, 40 and 50 cmH₂O. Vertical bars represent standard deviations. * p<0.05 for the prototype guayule latex cuff vs. the commercial HVLP cuffs at the same pressure.

FIG. 3 graphically illustrates the relationship between intracuff pressure and average measured transmitted pressure to the wall of the tracheal model.

### DETAILED DESCRIPTION OF VARIOUS EMBODIMENTS

The present disclosure provides for a two-layer endotracheal tube (ETT) cuff that forms no folds upon inflation within the trachea and shows no leakage across the cuff when tested. The disclosed ETT cuff shows marked and substantial advantages and performs better than four other high-volume low-pressure ETT cuffs known in the art. The disclosed two-layer leak-proof ETT cuff is highly advantageous in the prevention of VAP.

The present novel ETT cuff first utilizes a standard HVLP cuff that is then draped with a second, highly elastic cuff made of a low-protein guayule natural latex rubber, or other suitable elastomeric material, as described below, with a wall thickness of 50-60 μm. One half milliliter of gel is then introduced between these two cuffs.

The key physical attributes of the second layer (the draping layer) of the disclosed two-layer ETT cuff include its modulus and its tensile strength. The elastic modulus of a material represents the relative stiffness of the material within the elastic range and can be determined from a stress-strain curve by calculating the ratio of stress to strain. The tensile strength of a material is the maximum amount of tensile stress that it can be subjected to before failure.

According to the present disclosure, the modulus of the second or draping layer described above is approximately between 0.7 MPa (Mega Pascals or force per unit area) and 1.2 MPa at 100% elongation; and/or approximately 1.3 MPa to 2.2 MPa at 500% elongation. The tensile strength of the second or draping layer is approximately between 22 MPa and 28 MPa. A tensile strength in this approximate range is sufficient to ensure a crosslink density high enough to maintain water-tight integrity over time.

Other non-Hevea plant species that can be used to derive a non-Hevea source of elastomeric material, namely natural rubber latex, for use in the present disclosure include, but are not limited to, gopher plant (Euphorbia lathyris), mariola (Parthenium incanum), rabbitbrush (Chrysothanmus nauseosus), candelilla (Pedilanthus macrocarpus), Madagascar rubbervine (Cryptostegia grandiflora), milkweeds (Asclepias syriaca, speciosa, subulata, et al.), goldenrods (Solidago altissima, graminifolia, rigida, et al.), Russian dandelion (Taraxacum kok-saghyz), mountain mint (Pycnanthemum incanum), American germander (Teucreum canadense), and tall bellflower (Campanula americana). Many other plants which produce rubber and rubber-like hydrocarbons are known, particularly among the Asteraceae (Compositae), Euphorbiaceae, Campanulaceae, Labiatae, and Moraceae families.

Additionally, Hevea natural rubber latex may be used, namely latex derived from Hevea brasiliensis. According to the present disclosure Hevea latex is suitably compounded to achieve the low modulus required for the disclosed ETT cuff. Hevea, versus other synthetic elastomeric materials, is the most similar material to the disclosed guayule in terms of physical performance. Also, Hevea draped ETT cuffs could be made with an increased (larger) diameter to compensate for any additional stiffness in the Hevea film, i.e., a Hevea film that had a greater modulus than the guayule film.

Nitrile may also be suitably compounded to achieve a draping film of necessary integrity for the presently disclosed ETT cuff. Again, nitrile draped ETT cuffs could be made with an increased (larger) diameter to compensate for any additional stiffness in the nitrile film, i.e., a nitrile film that had a greater modulus than the guayule film.

Further, with adequate plasticization, polyvinylchloride (PVC) could be sufficiently softened to achieve a film with the low modulus needed for draping. Further, this would be convenient for manufacture, as the balloon on the tracheal tube cuff, in this alternate embodiment, could also be made mostly of plasticized PVC. Again, PVC draped ETT cuffs could be made with an increased (larger) diameter to compensate for any additional stiffness in the PVC film, i.e., a PVC film that had a greater modulus than the guayule film.

Additionally, synthetic polyisoprene (PI) may also be used with adequate plasticization. Through sufficient plasticization, the PI could be softened enough to achieve a film of reasonably low modulus. Further, the balloon on the tracheal tube cuff, in this alternate embodiment, could also be mostly made of PI. Again, PI-draped ETT cuffs could be made with an increased (larger) diameter to compensate for any additional stiffness in the PI film, i.e. a PI film that had a greater modulus than the guayule film.

In yet another embodiment, polyurethane (PU) may be use either with adequate plasticization and/or mix of hard and soft blocks. In either case, the PU could be softened enough to achieve a film of low enough modulus for draping. Further, the balloon on the tracheal tube cuff, in this alternate embodiment, could also be mostly made of PU. Again, PU-draped ETT cuffs could be made with an increased (larger) diameter to compensate for any additional stiffness in the PU film, i.e. a PU film that had a greater modulus than the guayule film.

Finally, block copolymers (BC) could also be softened enough to achieve a film with the necessary low modulus for draping. This could be accomplished either with adequate plasticization and/or a mix of hard and soft blocks. Again, the balloon on this embodiment could also be made mostly of plasticized BC, and BC draped ETT cuffs could be made with an increased (larger) diameter to compensate for any additional stiffness in the BC film.

The disclosed two-layer ETT cuff is manufactured by first covering a standard HVLP ETT cuff with a thin, high-compliance, low-protein guayule latex rubber cuff, or other elastomeric material, as described above. In using standard HVLP cuffs known in the art, low-inflation pressure is required to fill the trachea, invariably forming multiple longitudinal folds. Bacteria colonize oropharyngeal secretions and gastric contents can leak along the folds into the lower airways and the lungs which is a major risk factor for ventilator-associated pneumonia (VAP).

However, unlike standard HVLP cuffs, manufacturing the disclosed ETT cuffs with a second layer cuff that conforms to the specifications provided above, allows stretching of the compliant latex or elastomeric covering during inflation and eliminates folds, assuring a perfect seal. This two-layer cuff design is therefore beneficial in the prevention of ischemic damage to the trachea and the leakage of colonized secretions into the lower airways.

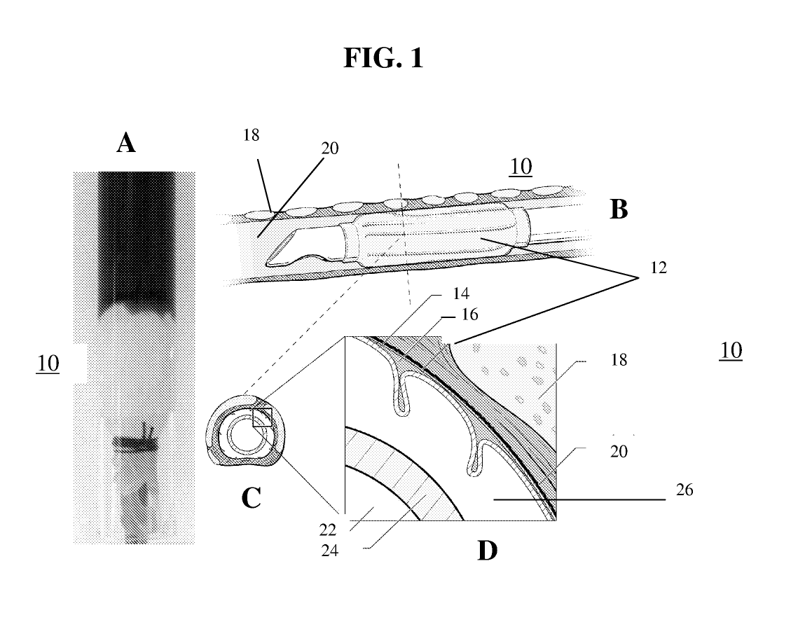

The two layer ETT cuff 10, shown in the picture in FIG. 1A and schematic in FIG. 1B, was manufactured with a guayule latex rubber second-layer cuff 12, cylindrical in shape (13 mm diameter), 50-60 μm thick. As shown in detail in FIGS. 1C and 1D, the second-layer guayule latex 12 is draped over the Mallinckrodt Hi-Lo Evac (ID: 8 mm) ETT cuff 14 or any other type of HVLP cuff known in the art. One half milliliter of sterile gel 16 (Surgilube, NY) or any other type of sterile gel, is introduced between the two cuffs to facilitate a homogeneous distribution of inflation pressure and to reduce friction between the two cuffs. The outer latex cuff was secured on both ends using silk ligatures.

FIGS. 1C and 1D also show the cuff in use with pressurized air 26 inside the cuff, as the cuff is inserted into the tracheal cartilage 18 and mucosa 20, and around the ETT lumen 22 and tracheal tube 24.

### Example 1

### Leakage Test

The guayule latex ETTs were tested and compared against four commercially available ETTs: Euromedical (Euromedical Industries, Malaysia), Mallinckrodt Hi-Lo Evac (Mallinckrodt, NY), Microcuff (Kimberly Clark Health Care, GA), Sheridan/CF (Hudson RCI, NC) for fluid leakage across the cuff. Tests were performed using vertically-positioned, cylindrical glass tubes, 20 cm long, of three internal diameters (16, 20 and 22 mm), matching the broad range of adult human tracheas. All ETTs (ID: 8 mm) were inflated at intracuff pressures of 20, 25, 30, 40, and 50 cmH₂O. A small reservoir was positioned below the model trachea to collect water leakage. Fifteen ml of water was poured above the cuff and observed until all water was collected or until the two-hour test period had ended.

Leakage is reported as average flow (ml/min) across the cuff, calculated by dividing the volume of water collected by either 120 minutes or the time at which all 15 ml of water had leaked. Three new ETTs of each type were tested by three different investigators. Three guayule latex prototype ETTs and three microcuff ETTs were similarly tested for 24 hours, using inflation pressure of 20 cmH₂O and a 20 mm diameter model trachea.

### Example 2

### Wall Pressure Measurement

Three new ETT cuffs of each type were inflated inside a glass tube (ID: 22 mm) at four pressures (10, 20, 30 and 40 cmH₂O). The pressure exerted against the internal wall of the glass tube was measured with a manometer connected to a flat, small (4 cm long, 1 cm wide), inelastic, partially inflated PVC cuff, inserted between the ETT cuff and the glass tube.

### Example 3

### Statistical Analysis

Non-parametric methods are used for comparisons of the guayule latex prototype ETT versus each of the four commercial ETTs. Since three observations per pressure/diameter block were measured, an F-approximation to the Friedman's test was obtained by using the generalized linear model method using the within block ranks to study the overall effect of the different cuffs. (Friedman's test is appropriate when there is only one response per treatment-block combination.)

Four pairwise comparisons of interest were performed only if the overall F-test was significant at p<0.05. The effect of pressure and diameter as well as their interactions with the ETT type on the leakage flow using ranks was also examined. A Bonferroni correction was applied for the four primary comparisons of interest. The volume of fluid leakage during the 24 hours test was compared with the Wilcoxon test. The statistical analysis was performed with R-project statistical software.

### Example 4

### Assessment of Folds and Leakage

The guayule latex prototype cuff inflated in all model tracheas showed no folds; however, folds always occurred with the four tested HVLP cuffs.

Table 1 below shows average leakage (ml/minute, mean ±s.d.) across each ETT cuff inflated at pressures of 20, 25, 30, 40 and 50 cmH₂O into model tracheas of 16, 20 and 22 mm in diameter. Both unadjusted and adjusted (pressure and diameter) analyses resulted in a p<0.0001 when comparing the guayule latex prototype to each of the other four ETT cuffs.

TABLE 1

| | 20 cmH₂0 | 25 cmH₂0 | 30 cmH₂0 | 40 cmH₂0 | 50 cmH₂0 |
|---|---|---|---|---|---|
| Sheridan\CF* | | | | | |
| 16 | 57.69 ± 32.50 | 44.08 ± 17.51 | 45.00 ± 22.81 | 21.52 ± 11.85 | 14.25 ± 5.86 |
| 20 | 47.79 ± 28.38 | 66.46 ± 74.59 | 37.99 ± 38.99 | 17.97 ± 9.65 | 13.88 ± 12.48 |
| 22 | 175.76 ± 237.77 | 37.32 ± 32.77 | 9.95 ± 8.53 | 2.71 ± 3.91 | 25.70 ± 37.90 |
| Euromedical* | | | | | |
| 16 | 9.74 ± 3.51 | 9.26 ± 4.56 | 8.98 ± 1.85 | 5.61 ± 2.08 | 5.66 ± 2.81 |
| 20 | 16.15 ± 8.59 | 9.23 ± 4.83 | 6.48 ± 5.32 | 4.62 ± 1.82 | 4.10 ± 2.09 |
| 22 | 8.97 ± 1.51 | 6.30 ± 0.88 | 5.27 ± 2.48 | 3.77 ± 2.28 | 3.51 ± 2.45 |
| Mallinckrodt | | | | | |
| Hi-Lo Evac* | | | | | |
| 16 | 12.94 ± 9.54 | 8.74 ± 5.56 | 6.63 ± 2.68 | 4.98 ± 2.29 | 3.37 ± 1.19 |
| 20 | 5.55 ± 1.71 | 5.20 ± 4.49 | 2.45 ± 1.05 | 2.14 ± 1.40 | 1.69 ± 1.92 |
| 22 | 6.76 ± 5.97 | 9.35 ± 6.80 | 2.59 ± 0.60 | 1.78 ± 0.48 | 1.13 ± 0.38 |
| Microcuff* | | | | | |
| 16 | 0.21 ± 0.19 | 0.23 ± 0.13 | 0.09 ± 0.02 | 0.04 ± 0.04 | 0.04 ± 0.03 |
| 20 | 0.17 ± 0.11 | 0.10 ± 0.07 | 0.06 ± 0.03 | 0.04 ± 0.05 | 0.02 ± 0.02 |
| 22 | 0.08 ± 0.03 | 0.05 ± 0.02 | 0.01 ± 0.02 | 0.00 ± 0.00 | 0.01 ± 0.01 |
| Guayule latex | | | | | |
| prototype* | | | | | |
| 16 | 0.00 ± 0.00 | 0.00 ± 0.00 | 0.00 ± 0.00 | 0.000083 ± 0.00014 | 0.00 ± 0.00 |
| 20 | 0.0028 ± 0.0041 | 0.00694 ± 0.0064 | 0.00 ± 0.00 | 0.00 ± 0.00 | 0.00 ± 0.00 |
| 22 | 0.00 ± 0.00 | 0.00083 ± 0.0014 | 0.00 ± 0.00 | 0.000083 ± 0.00014 | 0.00 ± 0.00 |

The average leakage in ml/min was 6.6×10⁻⁴+2.5×10⁻³ for the guayule latex prototype ETT, 7.3×10⁻²±9.3×10⁻² for the Microcuff, 5.0±4.7 for the Mallinckrodt Hi-Lo EVAC, 7.2±4.4 for the Euromedical and 41±69 for the Sheridan/CF. The primary analysis results showed that the overall test of equality among the five cuffs is significant (p<0.0001). In all four paired comparisons, guayule latex cuff outperformed the other ETT cuffs (Euromedical, Mallinckrodt, Microcuff, and Sheridan; p<0.0001).

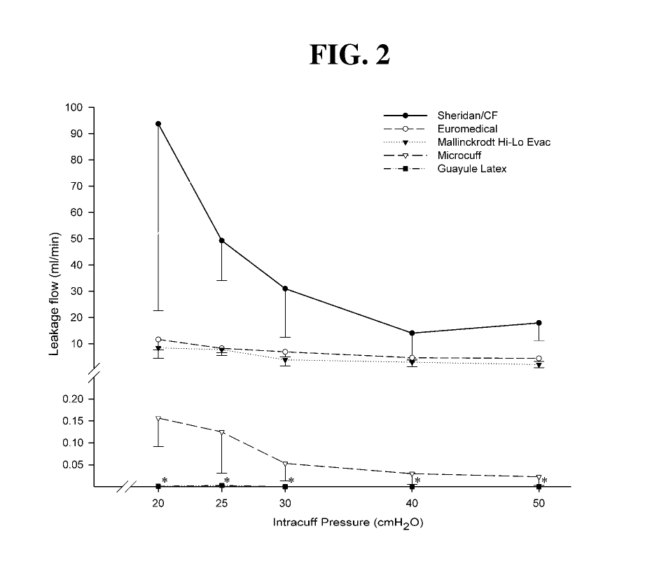

In a secondary analysis, adjusted for the significant variables (intracuff diameter and pressure), the guayule latex cuff also outperformed the other four cuffs (p<0.0001). The latter analysis suggests that the interaction of ETT brand and diameter was significant. However, all stratified by diameter analyses performed did not change the above conclusions. FIG. 2 shows average leakage flow as a function of the intracuff pressure. The latex prototype cuff performed significantly better (p<0.0001) than all others followed by Microcuff, Mallinckrodt Hi-Lo Evac, Euromedical, and Sheridan/CF.

The average volume of water leaked (ml) across the guayule latex prototype cuff and the microcuff was 0.9±5 and 14.1±2.2 (p=0.03) respectively.

### Example 5

### Tracheal Wall Pressure

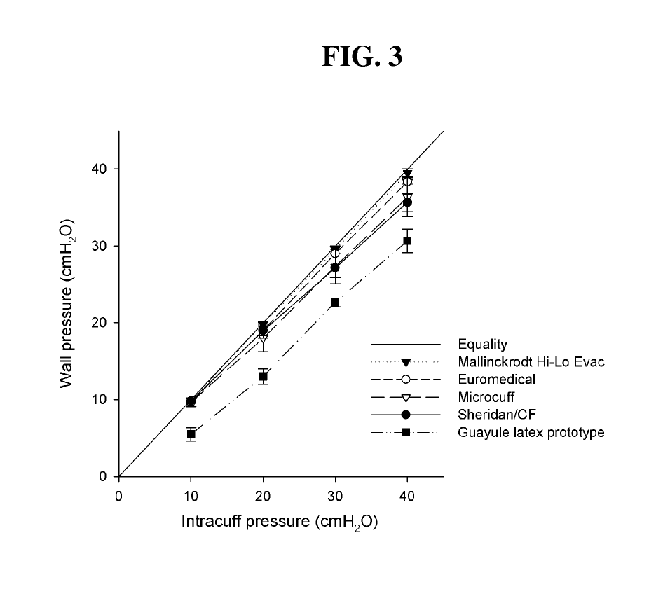

FIG. 3 shows the relationship between the intracuff pressure and the pressure transmitted to the model tracheal wall for each cuff. The guayule latex cuff exerted, on average, a wall pressure 7.0±1.9 cmH₂O (mean ±s.d.) lower than the intracuff pressure.

### Example 6

### In Vivo Results

Twenty pigs were used in the in vivo trial with fifteen control pigs using standard HVLP cuffs. The presently disclosed two-layer ETT cuff was inserted in the other five pigs. All fifteen control pigs contracted pneumonia while all of the five pigs with the disclosed two-layer cuff remained pneumonia free. Thus, further illustrating the pneumonia-preventing properties of the presently disclosed cuff compared with those cuffs that presently exist.

Therefore, a novel two-layer ETT cuff for the prevention of pneumonia is disclosed. The disclosed two-layer ETT cuff forms no folds when inflated in a model trachea and highly reduced, almost eliminated, fluid leakage at inflation pressure as low as 20 cmH₂O. In comparison, all commercial HVLP cuffs showed multiple folds and did not prevent fluid leakage, even at 50 cmH₂O.

The Microcuff cuff, made of thin (7 μm) polyurethane film, forms only small folds, and thus performed significantly better than the PVC HVLP cuffs. Dullenkopf previously tested the Microcuff and reported lower leakage compared to the presently disclosed two-layer cuff. The disclosed cuff was also tested in three model tracheas with different diameters vs. the single 20 mm diameter model trachea in Dullenkopf's study.

One advantage of the presently disclosed two-layer ETT cuff is that it utilizes a very thin outer layer of guayule latex to completely enclose a traditional HVLP cuff. Guayule latex rubber is highly elastic, tear resistant, compliant and requires low pressure to be stretched, allowing an almost complete transmission of the intracuff pressure to the tracheal wall. Therefore, the presently disclosed two-layer cuff creates a tight seal and prevents fluid leakage even at low pressures.

Although 1-8% of the population has hypersensitivity to Hevea latex proteins, the guayule latex material used in one embodiment of the disclosed cuff is does not contain the proteins known to cause reaction in latex sensitive people. Latex produced from the guayule shrub contains very little protein and no epitopes that cross-react with Type I latex allergy. Additionally, guayule latex also provides a strong barrier against blood-borne pathogens. The development of the disclosed two-layer leak-proof ETT is therefore a major step towards the prevention of VAP.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicants at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

What is claimed is:

1. An endotracheal tube cuff, comprising:
an inner layer comprising a high-volume low-pressure cuff;
an outer layer comprising an elastomeric material covering a portion of the inner layer; and
lubricant between the inner layer and the outer layer.

2. The endotracheal tube cuff of claim 1, wherein the elastomeric material comprises non-Hevea natural rubber latex.

3. The endotracheal tube cuff of claim 2, wherein the non-Hevea natural rubber latex is guayule natural rubber latex.

4. The endotracheal tube cuff of claim 1, wherein the elastomeric material comprises Hevea natural rubber latex.

5. The endotracheal tube cuff of claim 1, where in the elastomeric material is selected from a group consisting of nitrile, polyvinyl chloride, synthetic polyisoprene, polyurethane, and block copolymers.

6. The endotracheal tube cuff of claim 1, wherein the lubricant is sterile gel.

7. The endotracheal tube cuff of claim 1, further comprising a balloon.

8. The endotracheal tube cuff of claim 7, wherein the balloon substantially comprises a material selected from the group consisting of polyvinyl chloride, synthetic polyisoprene, polyurethane, and block copolymers.

9. The endotracheal tube cuff of claim 1, wherein the modulus of the outer layer at one hundred percent elongation is in a range of approximately 0.7 to 1.2 MPa.

10. The endotracheal tube cuff of claim 1, wherein the modulus of the outer layer at five hundred percent elongation is in a range of approximately 1.3 to 2.2 MPa.

11. The endotracheal tube cuff of claim 1, wherein the tensile strength of the outer layer is in a range of approximately 22 to 28 MPa.

12. A method for manufacturing an endotracheal tube cuff, comprising:
covering a portion of a high-pressure low-volume cuff with an elastomeric material.

13. The method of claim 12, wherein the elastomeric material comprises non-Hevea natural rubber latex.

14. The method of claim 13, wherein the non-Hevea natural rubber latex is guayule natural rubber latex.

15. The method of claim 12, further including introducing a lubricant in between the high-pressure low-volume cuff and the elastomeric material.

16. The method of claim 15, wherein the lubricant is sterile gel.

17. The method of claim 12, where in the elastomeric material is selected from a group consisting of nitrile, polyvinyl chloride, synthetic polyisoprene, polyurethane, and block copolymers.

18. The method of claim 17, further comprising softening the elastomeric material with adequate plasticization prior to covering the high-pressure low-volume cuff with the elastomeric material.

19. The method of claim 18, wherein softening the elastomeric material comprising reducing the modulus of the elastomeric material at one hundred percent elongation to a range of approximately 0.7 to 1.2 MPa.

20. The method of claim 18, claim 1, wherein softening the elastomeric material comprising reducing the modulus of the elastomeric material at five hundred percent elongation to a range of approximately 1.3 to 2.2 MPa.

21. The method of claim 12, wherein the tensile strength of the elastomeric material is in a range of approximately 22 to 28 MPa.

22. A method for preventing ventilator-associated pneumonia in a patient in need of mechanical ventilation, comprising:
inserting into the patient's trachea, an endotracheal tube cuff comprising an inner layer comprising a high-volume low-pressure cuff; an outer layer comprising an elastomeric material covering a portion of the inner layer; and lubricant between the inner layer and the outer layer.

23. The method of claim 22, wherein the endotracheal tube cuff has a diameter selected from the group consisting of 16 millimeters, 20 millimeters and 22 millimeters.
